(19) United States
(12) Patent Application Publication
(10) Pub. No.: US 2024/0203597 A1
(43) Pub. Date: Jun. 20, 2024

(54) PREDICTION DEVICE FOR PREDICTING INFORMATION RELATED TO PATIENT, OPERATION METHOD OF PREDICTION DEVICE, AND PROGRAM

(71) Applicant: FUJIFILM CORPORATION, Tokyo (JP)

(72) Inventors: Shotaro MISAWA, Tokyo (JP); Tomoko OHKUMA, Tokyo (JP); Ryuji KANO, Tokyo (JP); Tomoki TANIGUCHI, Tokyo (JP); Hirokazu YARIMIZU, Tokyo (JP); Kohei ONODA, Tokyo (JP)

(21) Appl. No.: 18/582,664

(22) Filed: Feb. 21, 2024

Related U.S. Application Data
(63) Continuation of application No. PCT/JP2022/031881, filed on Aug. 24, 2022.

(30) Foreign Application Priority Data
Aug. 25, 2021 (JP) 2021-137515

(51) Int. Cl. G16H 50/20; G16H 10/60

(52) U.S. Cl. CPC G16H 50/20; G16H 10/60

(57) ABSTRACT

A prediction device that predicts information related to a patient based on medical data of the patient, including: a processor; and a memory connected the processor, in which the processor being configured to execute: data set extraction processing of extracting M data sets by classifying pieces of medical data of plural patients into any one of M types of predetermined attributes, training data set generation processing of generating M training data sets related to the M types of attributes from the M data sets, similarity calculation processing of calculating, for each pair of the M types of attributes, a similarity between the attributes, training processing of training one or plural machine learning models based on the similarity between the attributes by using the M training data sets, and prediction processing of causing the one or plural machine learning models to predict the information related to the patient.

### CROSS-REFERENCE TO RELATED APPLICATIONS

This application is a continuation of International Application No. PCT/JP2022/031881, filed on Aug. 24, 2022, which claims priority from Japanese Application No. 2021-137515, filed on Aug. 25, 2021. The entire disclosure of each of the above applications is incorporated herein by reference.

### BACKGROUND

### Technical Field

The present disclosure relates to a prediction device for predicting information related to a patient, an operation method of the prediction device, and a program.

### Related Art

In a medical field, in a case of predicting information related to a patient, a machine learning model specialized for a specific attribute (for example, a disease) has been developed. For example, JP 2018-36900 A discloses a machine learning model specialized in predicting a prognosis of a patient with severe heart failure.

Usually, training data sets of a machine learning model specialized for a specific disease are created from pieces of medical data of patients who suffered from the specific disease in the past. In the example of JP2018-36900A, training data sets of a machine learning model that predicts a prognosis of a patient with severe heart failure are created from pieces of medical data of patients who suffered from severe heart failure in the past. However, in a case where an amount of available medical data is small, there is a possibility that an amount of training data sets required for a machine learning model specialized for a specific disease to achieve a desired prediction accuracy cannot be obtained.

### SUMMARY

According to the present disclosure, there is provided a prediction device capable of improving a prediction accuracy as compared with the related art even in a case where, in training of a machine learning model specialized for a specific attribute (for example, a disease), a sufficient amount of training data sets related to the specific attribute is not obtained.

According to a first aspect of the present disclosure, there is provided a prediction device that predicts information related to a patient based on medical data of the patient, the prediction device including: a processor; and a memory connected to or built in the processor, in which the processor is configured to execute data set extraction processing of extracting M data sets by classifying pieces of medical data of a plurality of patients into any one of M types of predetermined attributes, training data set generation processing of generating M training data sets related to the M types of attributes from the M data sets, similarity calculation processing of calculating, for each pair of the M types of attributes, a similarity between the attributes, training processing of training one or a plurality of machine learning models based on the similarity between the attributes by using the M training data sets, and prediction processing of causing the one or the plurality of machine learning models to predict the information related to the patient.

According to a second aspect of the present disclosure, in the first aspect, the M types of attributes may be M types of diseases or M types of medical departments, and the similarity between the attributes may be a similarity between the diseases or a similarity between the medical departments.

According to a third aspect of the present disclosure, in the second aspect, the similarity between the attributes may be calculated based on at least one of a distance between organs, a distance on a circulatory system, or a metastasis route of a cancer.

According to a fourth aspect of the present disclosure, in the second aspect, the similarity between the attributes may be calculated based on information included in the data set.

According to a fifth aspect of the present disclosure, in the fourth aspect, the information included in the data set may include at least one of a symptom, an examination result, an examination image, an age of a patient, an attending physician, a medical department, a disease, a treatment, a medication, a candidate for differential diagnosis, or the number of co-occurrences.

According to a sixth aspect of the present disclosure, in any one of the first aspect to the fifth aspect, the one or the plurality of machine learning models may be a single machine learning model, the processor may be configured to further execute order determination processing of determining an order of the M types of attributes based on a similarity between a prediction target and the attribute, and the processor may be configured to, in the training processing, retrain the single machine learning model by using the M training data sets related to the M types of attributes in order according to the order of the M types of attributes.

According to a seventh aspect of the present disclosure, in the sixth aspect, the processor may be configured to, in the order determination processing, set an attribute corresponding to the prediction target as an M-th attribute, and determine an order of the other M−1 attributes in descending order of a similarity between the M-th attribute and the attribute.

According to an eighth aspect of the present disclosure, in the seventh aspect, the processor may be configured to, in the training processing, set N to a natural number between 1 and M−1, train the untrained single machine learning model by using the training data set related to an N-th attribute, retrain the trained single machine learning model by using the training data set related to an (N+1)-th attribute, and perform retraining in order to the M-th attribute.

According to a ninth aspect of the present disclosure, in any one of the first aspect to the fifth aspect, the one or the plurality of machine learning models may include a plurality of machine learning models, the processor may be configured to further execute common layer addition/combination processing of adding a common layer and combining the common layer on the plurality of machine learning models based on a similarity between a prediction target and the attribute, and in the training processing, training of the common layer may be performed by using training data sets related to a plurality of attributes.

According to a tenth aspect of the present disclosure, in any one of the first aspect to the fifth aspect, the one or the plurality of machine learning models may include a first machine learning model related to a first attribute and a second machine learning model related to a second attribute, the processor may be configured to execute constraint generation processing of generating a constraint that a similarity between the attributes and a similarity between configurations of the first machine learning model and the second machine learning model have a positive correlation, and in the training processing, training of the first machine learning model and the second machine learning model may be performed in consideration of the constraint.

According to an eleventh aspect of the present disclosure, there is provided an operation method of a prediction device that predicts information related to a patient based on medical data of the patient, the method including: a step of extracting M data sets by classifying pieces of medical data of a plurality of patients into any one of M types of predetermined attributes; a step of generating M training data sets related to the M types of attributes from the M data sets; a step of calculating, for each pair of the M types of attributes, a similarity between the attributes; a step of training one or a plurality of machine learning models based on the similarity between the attributes by using the M training data sets; and a step of causing the one or the plurality of machine learning models to predict the information related to the patient.

According to a twelfth aspect of the present disclosure, there is provided a program for predicting information related to a patient based on medical data of the patient, the program causing a computer to execute a process including: a step of extracting M data sets by classifying pieces of medical data of a plurality of patients into any one of M types of predetermined attributes; a step of generating M training data sets related to the M types of attributes from the M data sets; a step of calculating, for each pair of the M types of attributes, a similarity between the attributes; a step of training one or a plurality of machine learning models based on the similarity between the attributes by using the M training data sets; and a step of causing the one or the plurality of machine learning models to predict the information related to the patient.

### BRIEF DESCRIPTION OF THE DRAWINGS

FIG. 1 is a diagram illustrating a schematic configuration of a prognosis prediction system according to an exemplary embodiment 1.

FIG. 2 is a block diagram illustrating a hardware configuration of a prediction server according to the exemplary embodiment 1.

FIG. 3 is a diagram illustrating a functional configuration of the prediction server according to the exemplary embodiment 1.

FIG. 4 is a diagram illustrating an example of pieces of medical data of a plurality of patients according to the exemplary embodiment 1.

FIG. 5 is a diagram illustrating an example of three data sets according to the exemplary embodiment 1.

FIG. 6 is a diagram illustrating an example of three training data sets according to the exemplary embodiment 1.

FIG. 7 is a diagram illustrating an example of a similarity table according to the exemplary embodiment 1.

FIG. 8 is a diagram illustrating an example of an order of retraining according to the exemplary embodiment 1.

FIG. 9 is a diagram illustrating an example of medical data according to the exemplary embodiment 1.

FIG. 10 is a flowchart illustrating an operation of the prediction server according to the exemplary embodiment 1 in a training phase.

FIG. 11 is a diagram illustrating a functional configuration of a prediction server according to an exemplary embodiment 2.

FIG. 12 is a diagram illustrating an example of a similarity table according to the exemplary embodiment 2.

FIG. 13 is a diagram illustrating an example of a common layer according to the exemplary embodiment 2.

FIG. 14 is a diagram illustrating a functional configuration of a prediction server according to an exemplary embodiment 3.

FIG. 15 is a diagram illustrating an example of training with a constraint according to the exemplary embodiment 3.

### DESCRIPTION

Hereinafter, in exemplary embodiments of the present disclosure, an example in which a technical idea of the present disclosure is applied to a prognosis prediction system that predicts a prognosis of an inpatient based on medical data of the inpatient will be described with reference to the accompanying drawings. Here, a scope to which the technical idea of the present disclosure can be applied is not limited thereto. Further, in addition to the disclosed exemplary embodiments, various forms that can be implemented by those skilled in the art are within the scope of the claims.

### Exemplary Embodiment 1

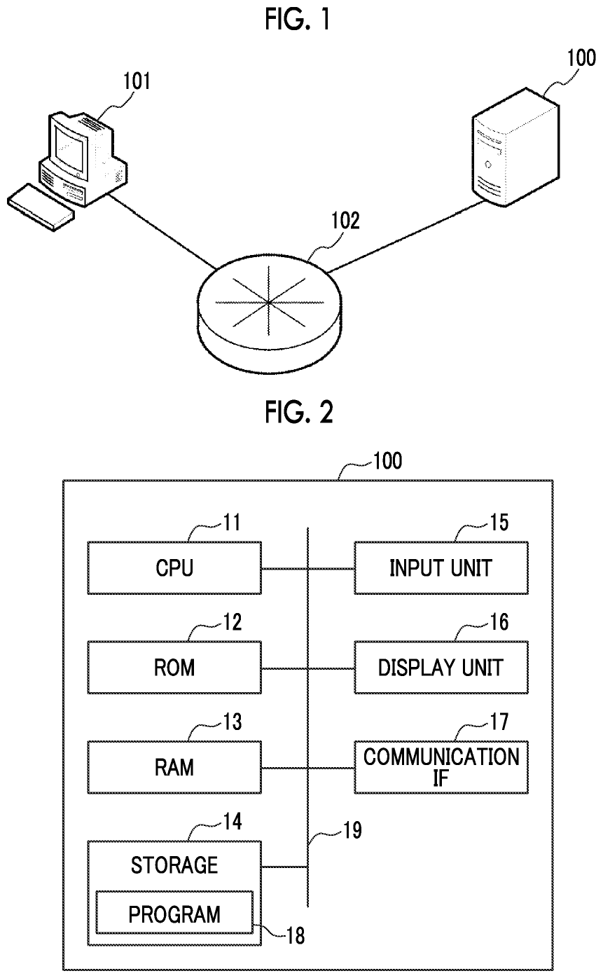

FIG. 1 is a diagram illustrating a schematic configuration of a prognosis prediction system according to an exemplary embodiment 1 of the present disclosure. The prognosis prediction system includes a prediction server 100, a user terminal 101, and a communication line 102 that connects the prediction server 100 and the user terminal 101 to each other for communication.

The prediction server 100 predicts a prognosis of a patient based on medical data of the patient that is transmitted from the user terminal 101 via the communication line 102. The prediction server 100 returns a predicted prognosis of the patient to the user terminal 101 via the communication line 102.

The user terminal 101 is a well-known personal computer. The communication line 102 is the Internet, an intranet, or the like. The communication line 102 may be a wired line or a wireless line. In addition, the communication line 102 may be a dedicated line or a public line.

FIG. 2 is a block diagram illustrating a hardware configuration of the prediction server 100. The prediction server 100 includes a central processing unit (CPU) 11, a read only memory (ROM) 12, a random access memory (RAM) 13, a storage 14, an input unit 15, a display unit 16, and a communication interface 17. The hardware components are communicably connected to each other via a bus 19.

The CPU 11 is a central arithmetic processing unit. The CPU 11 reads a program stored in the ROM 12 or the storage 14, and executes the program by using the RAM 13 as a work area. In the present exemplary embodiment 1, a program 18 for predicting a prognosis of a patient based on medical data of the patient is stored in the storage 14.

The ROM 12 stores various programs and various types of data. The RAM 13 as a work area temporarily stores the program or the data. The storage 14 is configured with a storage device such as a hard disk drive (HDD), a solid state disk (SSD), or a flash memory, and stores various programs including an operating system and various types of data.

The input unit 15 is configured with a mouse, a keyboard, and the like, and is used in a case where a user inputs data to the prediction server 100.

The display unit 16 is, for example, a liquid crystal display panel, and is used in a case where the prediction server 100 presents information to the user. Note that the display unit 16 and the input unit 15 may be implemented in common by adopting a touch-panel-type liquid crystal display panel.

The communication interface 17 is an interface that allows the prediction server 100 to perform communication with another device such as the user terminal 101. As a standard of the communication interface 17, for example, Ethernet (registered trademark), a fiber distributed data interface (FDDI), or Wi-Fi (registered trademark) can be adopted.

### (Functional Configuration of Prediction Server 100)

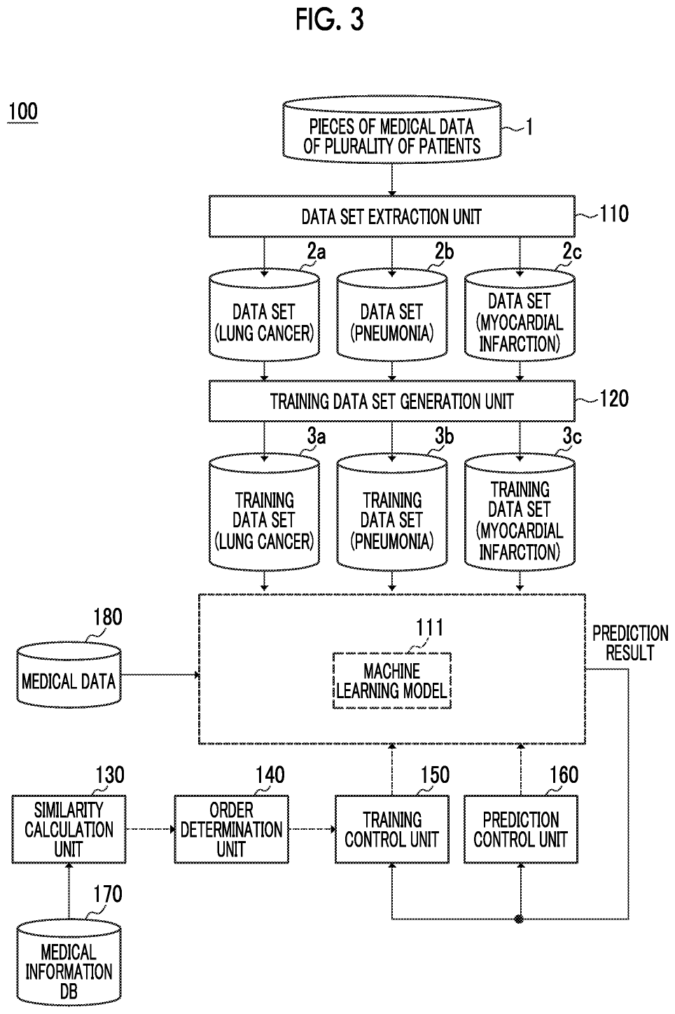

FIG. 3 is a diagram illustrating a functional configuration of the prediction server 100 according to the present exemplary embodiment 1. The prediction server 100 includes, as functional components, a data set extraction unit 110, a training data set generation unit 120, a similarity calculation unit 130, an order determination unit 140, a training control unit 150, and a prediction control unit 160. These functional components are implemented by the CPU 11 executing a program 18 stored in the storage 14.

In an operation phase, the prediction server 100 aims to predict a hospitalization period of a lung cancer patient based on medical data of the lung cancer patient. In a training phase of the prediction server 100, training of a machine learning model 111 is performed. The machine learning model 111 is a deep learning model based on a neural network, and includes an input layer, one or a plurality of interlayers, and an output layer. In a case where training of the machine learning model 111 is performed, not only medical data of lung cancer patients in the past but also medical data of patients with other diseases in the past are used together. The machine learning model 111 is untrained in an initial state. As an example, the untrained machine learning model is stored in the storage 14. In addition, the trained machine learning model 111 is also stored in the storage 14.

The prediction server 100 extracts a plurality of data sets 2a to 2c by classifying pieces of medical data 1 of a plurality of patients for each “disease” as an attribute, and generates training data sets 3a to 3c related to each disease from the data sets 2a to 2c. The generated training data sets 3a to 3c are used in a training phase in which the untrained machine learning model 111 is retrained in order. Note that, as the attribute, for example, a “medical department” may be considered instead of “disease” described above.

In the training phase, the prediction server 100 calculates a similarity between diseases for each pair of diseases, and determines an order of the diseases based on the similarity between diseases. The prediction server 100 retrains the untrained machine learning model 111 step by step by using the training data sets 3a to 3c related to cach disease in order according to the order of the diseases. This learning method is so-called curriculum learning.

In an operation phase of the prediction server 100, medical data 180 of a lung cancer patient whose a hospitalization period is desired to be predicted is input to the trained machine learning model 111. The trained machine learning model 111 predicts a hospitalization period of the patient based on the medical data 180 of the patient.

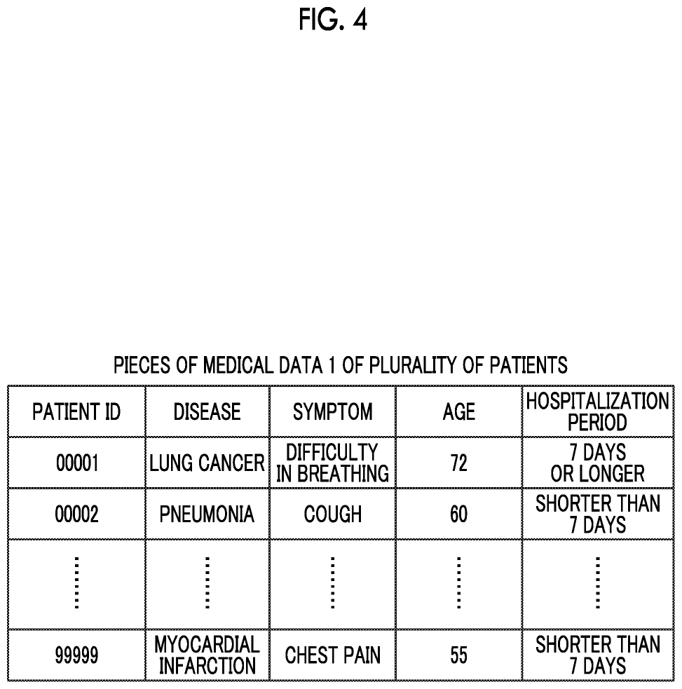

FIG. 4 is a diagram illustrating an example of pieces of medical data 1 of a plurality of patients in the present exemplary embodiment 1. The medical data of each patient includes a patient identifier (ID) and information on “disease”, “symptom”, “age”, and “hospitalization period” of the patient.

The “disease” takes any value of “lung cancer”, “pneumonia”, or “myocardial infarction” in the present example. In the present example, “symptom” takes any value of “cough”, “chest pain”, or “difficulty in breathing”. The “age” takes an integer value from “0” to “130” in the present example. The “hospitalization period” takes any value of “shorter than 7 days” or “7 days or longer” in the present example.

### (Data Set Extraction Unit 110)

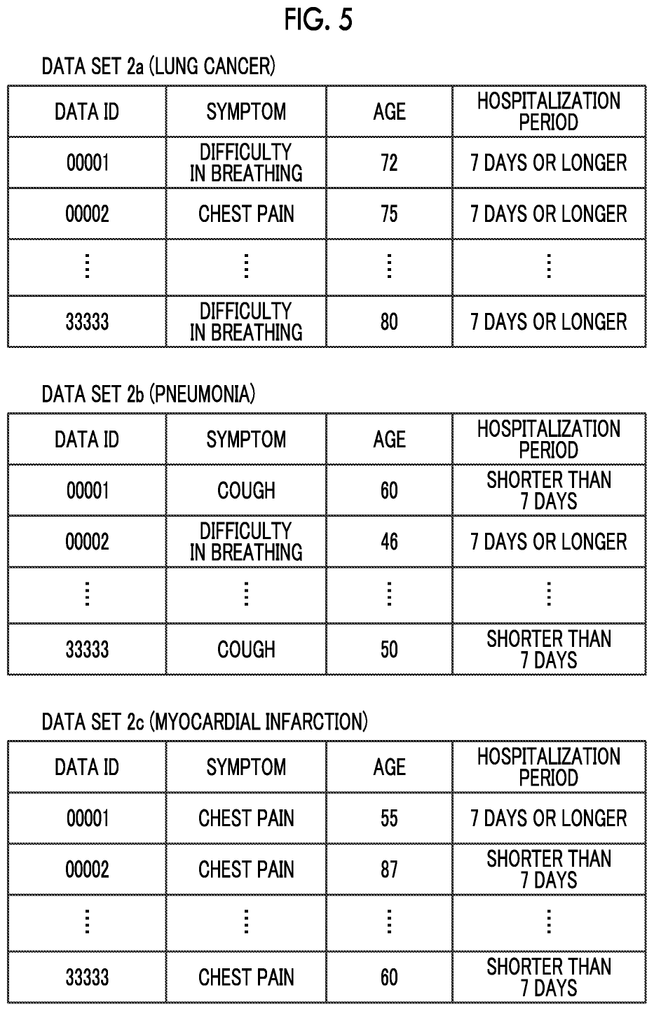

The data set extraction unit 110 classifies the pieces of medical data 1 of the plurality of patients into any one of three types of diseases, and extracts three data sets 2a to 2c. The data set 2a is a data set related to “lung cancer”. The data set 2b is a data set related to “pneumonia”. The data set 2c is a data set related to “myocardial infarction”. FIG. 5 is a diagram illustrating an example of three data sets 2a to 2c.

### (Training Data Set Generation Unit 120)

The training data set generation unit 120 generates training data sets 3a to 3c related to cach disease from the three data sets 2a to 2c. The training data set 3a is a training data set related to “lung cancer”. The training data set 3b is a training data set related to “pneumonia”. The training data set 3c is a training data set related to “myocardial infarction”.

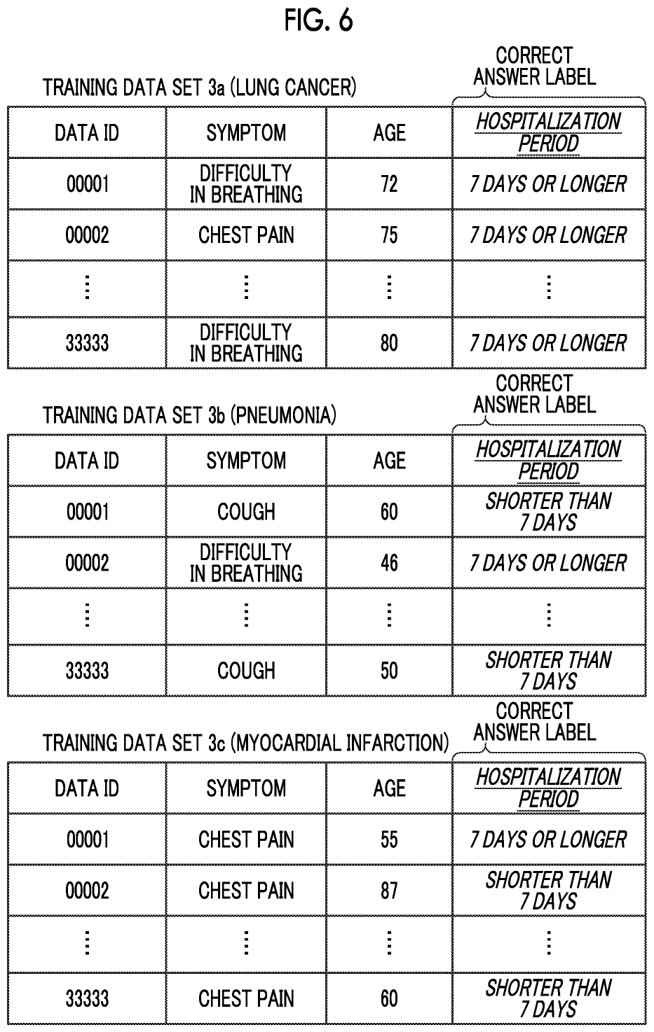

FIG. 6 is a diagram illustrating an example of three training data sets 3a to 3c. Each training data set includes a data ID, information of “symptom” and “age”, and “hospitalization period” as a correct answer label. In the data sets 2a to 2c, “hospitalization period” is one of pieces of information included in the data set. On the other hand, in the training data sets 3a to 3c, “hospitalization period” is treated as a correct answer label.

### (Similarity Calculation Unit 130)

The similarity calculation unit 130 calculates a similarity between diseases for each pair of diseases. In a first example, the similarity between diseases is calculated based on the information included in the data sets 2a to 2c. For example, the similarity between diseases is calculated based on “symptom” included in the data sets 2a to 2c. In general, examples of information that can be included in the data sets extracted from the pieces of medical data 1 of the plurality of patients include “symptom”, “examination result”, “examination image”, “age of patient”, “attending physician”, “medical department”, “disease”, “candidate for differential diagnosis”, “number of co-occurrences”, and the like.

In a second example, the similarity between diseases is calculated based on information that cannot be included in the data sets 2a to 2c. For example, the similarity between diseases is calculated based on “a distance between organs”, “a distance on a circulatory system”, “a metastasis route of a cancer”, or the like. In this case, the similarity calculation unit 130 accesses a medical information DB 170, acquires the information, and calculates the similarity between diseases.

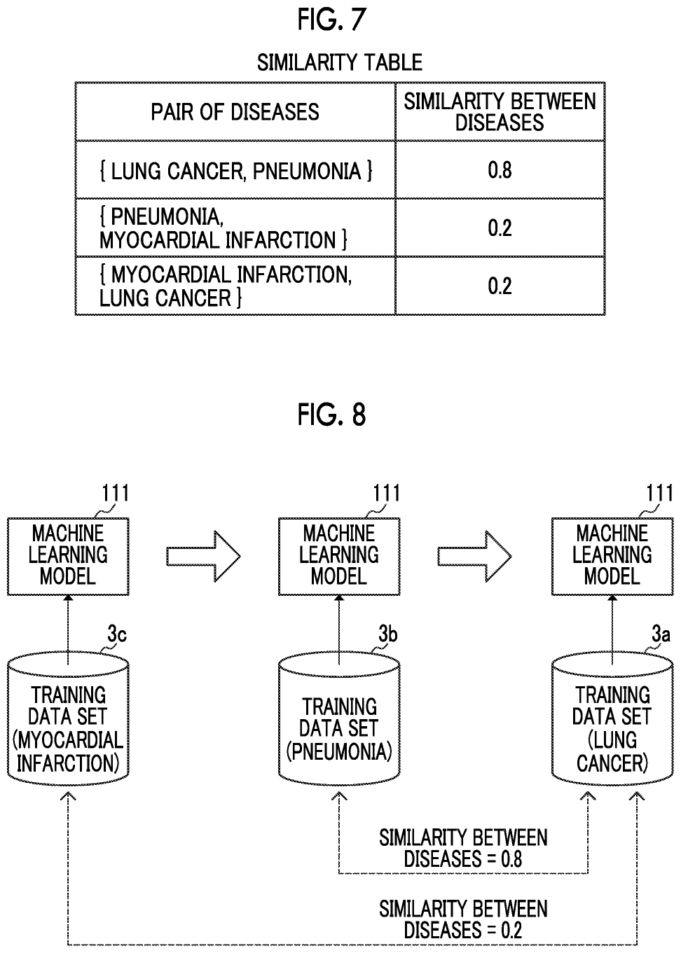

The similarity calculation unit 130 calculates the similarity between diseases for each pair of diseases, and creates a similarity table as illustrated in FIG. 7. In the example of FIG. 7, the similarity between diseases has a value equal to or higher than 0 and equal to or lower than 1. The similarity between diseases for a pair of “lung cancer” and “pneumonia” is highest, 0.8, the similarity between diseases for a pair of “pneumonia” and “myocardial infarction” is 0.2, and the similarity between diseases for a pair of “myocardial infarction” and “lung cancer” is also 0.2.

### (Order Determination Unit 140)

The order determination unit 140 determines an order of the diseases based on the similarity between diseases for a pair of a prediction target and each disease. Specifically, the order determination unit 140 sets types of diseases to M, sets a disease corresponding to a prediction target to an M-th disease, and determines the order of other M−1 diseases in descending order of the similarity with the M-th disease.

As described above, in the present exemplary embodiment 1, the prediction target is the hospitalization period of the lung cancer patient. In this case, the order determination unit 140 sets “lung cancer” as a third disease, and determines the order of “pneumonia” and “myocardial infarction” in descending order of the similarity with “lung cancer”. Specifically, “myocardial infarction” having the lowest similarity with “lung cancer” is set as a first disease, and “pneumonia” having the second lowest similarity with “lung cancer” is set as a second disease. Therefore, the order of the discases is determined to be the order of “myocardial infarction”, “pneumonia”, and “lung cancer”.

### (Training Control Unit 150)

The training control unit 150 retrains the machine learning model 111 step by step by using the training data sets related to each disease in order according to the order of the diseases that is determined by the order determination unit 140. Specifically, as illustrated in FIG. 8, the training control unit 150 first trains the untrained machine learning model 111 by using the training data set 3c related to “myocardial infarction” which is a first disease. Next, the training control unit 150 retrains the trained machine learning model 111 by using the training data set 3b related to “pneumonia” which is a second disease. Finally, the training control unit 150 retrains the trained machine learning model 111 by using the training data set 3a related to “lung cancer” which is a third disease.

### (Prediction Control Unit 160)

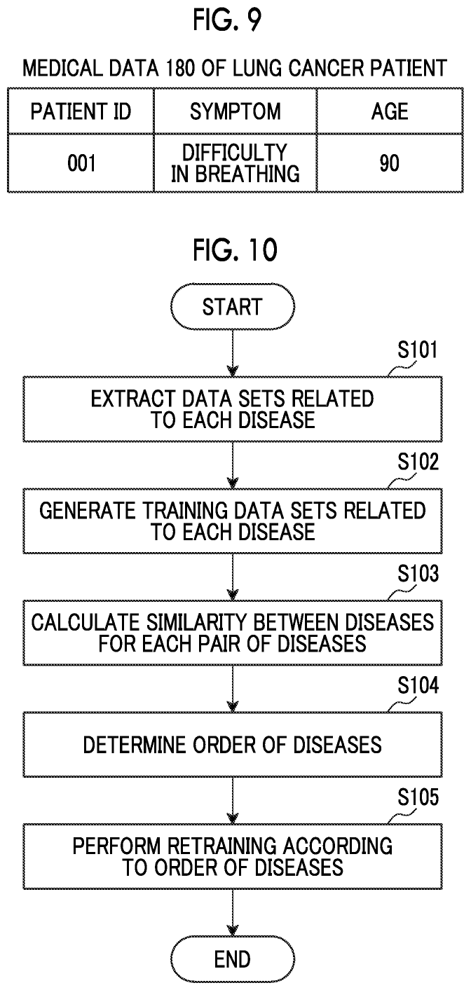

The prediction control unit 160 inputs the medical data 180 of the lung cancer patient for which the hospitalization period is desired to be predicted to the trained machine learning model 111. FIG. 9 is a diagram illustrating an example of the medical data 180. The medical data 180 includes a patient ID, and pieces of information of “symptom” and “age”. The trained machine learning model 111 predicts and outputs a hospitalization period of the lung cancer patient based on “symptom” and “age” included in the medical data 180.

### (Operation of Prediction Server 100 in Training Phase)

Next, an operation of the prediction server 100 in a training phase according to the present exemplary embodiment 1 will be described with reference to a flowchart in FIG. 10.

In step S101 of FIG. 10, the data set extraction unit 110 classifies pieces of medical data 1 of a plurality of patients for each disease, and extracts data sets 2a to 2c related to cach discasc.

In step S102, the training data set generation unit 120 generates training data sets 3a to 3c related to cach disease from the data sets 2a to 2c related to each disease.

In step S103, the similarity calculation unit 130 calculates a similarity between diseases for each pair of diseases.

In step S104, the order determination unit 140 determines an order of the diseases based on the similarity between diseases for a pair of a prediction target and each disease.

In step S105, the training control unit 105 retrains the machine learning model 111 by using the training data sets related to each disease in order according to the order of the diseases that is determined in step S104.

By processing described above, the machine learning model 111 is a model specialized in predicting the hospitalization period of the lung cancer patient.

In the training phase, the training data sets related to each disease are used in order. On the other hand, the training data sets used in the later stages have a greater influence on the characteristics of the final machine learning model 111. Therefore, the training data set 3a related to the prediction target, that is, related to “lung cancer” corresponding to prediction of the hospitalization period of the lung cancer patient is lastly used, and the training data set 3c related to “myocardial infarction” having the lowest similarity with “lung cancer” is initially used. Thereby, even in a case where a sufficient amount of training data sets related to “lung cancer” cannot be obtained, by using the training data sets related to “pneumonia” and “myocardial infarction”, it is possible to secure an amount of the training data sets required for the machine learning model 111 to obtain a desired prediction accuracy.

Here, in a case where a training data set related to a disease that has a very low similarity with the disease corresponding to the prediction target is used, an adverse effect may be given in training of the machine learning model 111. Therefore, in a case where the total number of the training data sets is M, N is set to a natural number between 1 and M−1, and training of the machine learning model 111 may be started from a training data set related to an N-th disease. In other words, the first to (N−1)-th training data sets may not be used. Thereby, it is possible to avoid adversely affecting training of the machine learning model 111.

As described above, the prediction server 100 according to the exemplary embodiment 1 of the present disclosure functions as a prediction device that predicts information related to a patient based on medical data of the patient. The prediction device calculates a similarity between diseases for each pair of a plurality of diseases, and determines an order of the diseases based on the similarity between diseases for a pair of a prediction target and each disease. The prediction device retrains the single machine learning model by using the training data sets related to each disease in order according to the order of the diseases that is determined in this manner. Thereby, even in a case where a sufficient amount of training data sets related to a specific disease cannot be obtained in training the machine learning model specialized for the specific disease, it is possible to improve the prediction accuracy as compared with the related art.

As described above, the prediction server 100 according to the exemplary embodiment 1 of the present disclosure functions as a prediction device that predicts information related to a patient based on medical data of the patient. The prediction device calculates a similarity between diseases for each pair of a plurality of diseases, and determines an order of the diseases based on the similarity between diseases for a pair of a prediction target and each disease. The prediction device retrains the single machine learning model by using the training data sets related to each disease in order according to the order of the diseases that is determined in this manner. Thereby, even in a case where a sufficient amount of training data sets related to a specific attribute cannot be obtained in training the machine learning model specialized for the specific attribute indicating a specific disease as an example, it is possible to improve the prediction accuracy as compared with the related art.

Further, as compared with a machine learning model that is trained by using all available training data sets without being limited to a specific disease, the machine learning model that is trained as described above can obtain a higher prediction accuracy for a specific disease.

### Exemplary Embodiment 2

Next, the prediction server 200 according to an exemplary embodiment 2 of the present disclosure will be described. Note that, in the following description, components that are the same as or similar to those in the exemplary embodiment 1 are denoted by the same reference numerals and a detailed description of the components will be omitted.

### (Prediction Server 200)

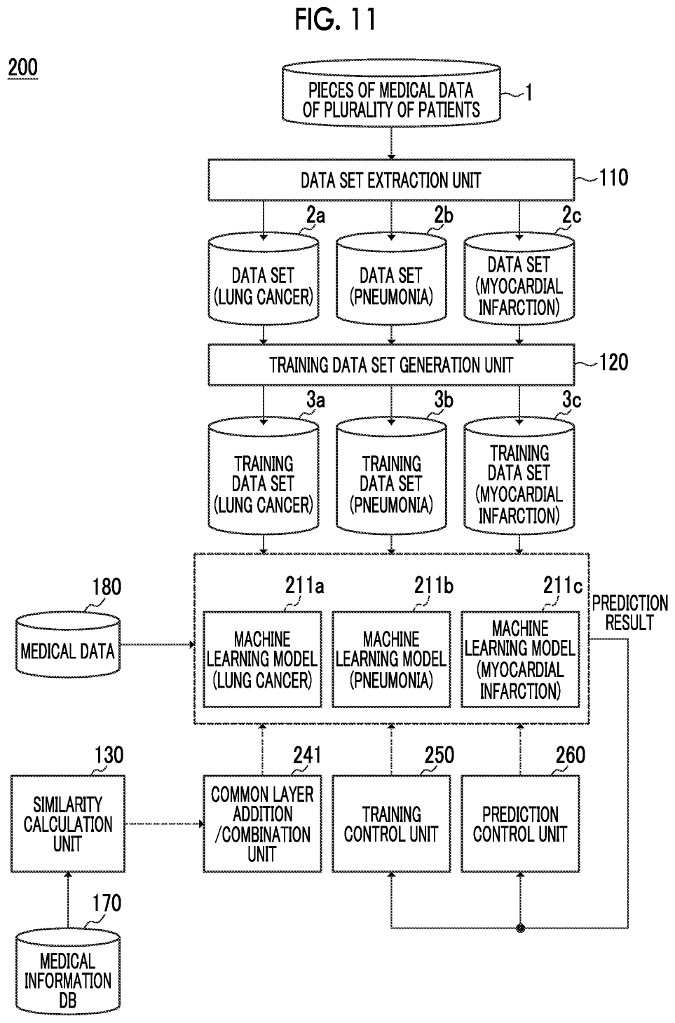

FIG. 11 is a diagram illustrating a configuration of a prediction server 200 according to an exemplary embodiment 2 of the present disclosure. The prediction server 200 includes a common layer addition/combination unit 241 instead of the order determination unit 140 included in the prediction server 100 according to the exemplary embodiment 1. Further, in the prediction server 200, the training control unit 150 and the prediction control unit 160 included in the prediction server 100 according to the exemplary embodiment 1 are respectively replaced with a training control unit 250 and a prediction control unit 260.

In addition, the prediction server 200 includes machine learning models 211a to 211c specialized for each disease. Specifically, the machine learning model 211a is a model specialized in predicting a hospitalization period of a lung cancer patient. The machine learning model 211b is a model specialized in predicting a hospitalization period of a pneumonia patient. The machine learning model 211c is a model specialized in predicting a hospitalization period of a myocardial infarction patient.

Further, even in the present exemplary embodiment 2, a prediction target is a hospitalization period of a lung cancer patient. Therefore, the machine learning model 211a that predicts a hospitalization period of a lung cancer patient is a machine learning model corresponding to the prediction target.

### (Common Layer Addition/Combination Unit 241)

The common layer addition/combination unit 241 adds a common layer and combines the common layer on the machine learning models 211a to 211c based on a similarity between diseases for a pair of a prediction target and each disease. Specifically, the common layer addition/combination unit 241 sets the machine learning model 211a corresponding to the prediction target as a reference, adds an interlayer including layers, of which the number is proportional to the similarity between the corresponding diseases, to a pair of the machine learning models 211a and 211b and a pair of the machine learning models 211a and 211c, and combines the interlayer that can be combined.

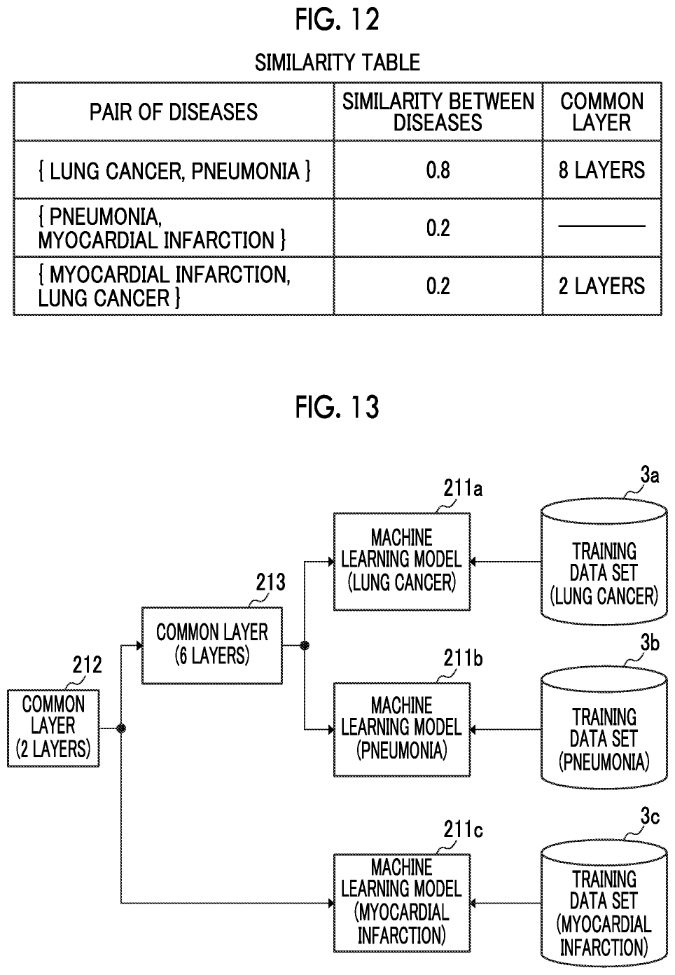

Specifically, for example, in a case where the similarity between diseases for each pair of the diseases is as illustrated in a middle column of FIG. 12, the common layer addition/combination unit 241 adds a common layer and combines the common layer on the machine learning models 211a to 211c as follows.

First, for a pair of the machine learning model 211a specialized for “lung cancer” corresponding to the prediction target and the machine learning model 211b specialized for “pneumonia”, the similarity between “lung cancer” and “pneumonia” is 0.8. Thus, for example, a common layer including 8 layers, which are obtained by a floor function [0.8×10], is added to the pair. Thereby, “8 layers” is described in the corresponding right column of FIG. 12.

Next, for a pair of the machine learning model 211a specialized for “lung cancer” corresponding to the prediction target and the machine learning model 211c specialized for “myocardial infarction”, the similarity between “lung cancer” and “myocardial infarction” is 0.2. Thus, for example, a common layer including 2 layers, which are obtained by a floor function [0.2×10], is added to the pair. Thereby, “2 layers” is described in the corresponding right column of FIG. 12.

Finally, by combining the common layers each of which includes 2 layers and which are common to the pair of the machine learning models 211a and 211b and the pair of the machine learning models 211a and 211c, a single common layer 212 including 2 layers is set, and the number of the layers of the common layer 213 for the pair of the machine learning models 211a and 211b is set to 6 layers, which is obtained by 8−2.

By the operation described above, as illustrated in FIG. 13, a common layer 212 and a common layer 213 are added.

### (Training Control Unit 250)

The training control unit 250 trains the common layer 212, the common layer 213, and the machine learning model 211a by using the training data set 3a related to “lung cancer” according to an error backward propagation method.

Similarly, the training control unit 250 trains the common layer 212, the common layer 213, and the machine learning model 211b by using the training data set 3b related to “pneumonia” according to an error backward propagation method.

Similarly, the training control unit 250 trains the common layer 212 and the machine learning model 211c by using the training data set 3c related to “myocardial infarction” according to an error backward propagation method.

As described above, the common layer 212 includes a relatively small number of layers, that is, 2 layers, reflecting a relatively low similarity among “lung cancer”, “pneumonia”, and “myocardial infarction”. On the other hand, training is performed by using all the training data sets 3a to 3c related to “lung cancer”, “pneumonia”, and “myocardial infarction”. On the other hand, the common layer 213 includes a relatively large number of layers, that is, 6 layers, reflecting a relatively high similarity between “lung cancer” and “pneumonia”. On the other hand, training is performed by using only the training data sets 3a and 3b related to “lung cancer” and “pneumonia”. In this manner, training is performed by using as many training data sets as possible in consideration of the similarity between diseases.

### (Prediction Control Unit 260)

In a case where it is desired to predict a hospitalization period of a lung cancer patient, the prediction control unit 260 inputs the medical data 180 of the lung cancer patient to the machine learning model 211a specialized for “lung cancer” via the common layer 212 and the common layer 213.

In addition, in a case where it is desired to predict a hospitalization period of a pneumonia patient, the prediction control unit 260 inputs the medical data 180 of the pneumonia patient to the machine learning model 211b specialized for “pneumonia” via the common layer 212 and the common layer 213.

In addition, in a case where it is desired to predict a hospitalization period of a patient with myocardial infarction, the prediction control unit 260 inputs the medical data 180 of the patient with myocardial infarction to the machine learning model 211c specialized for “myocardial infarction” via only the common layer 212.

As described above, the prediction server 200 according to the exemplary embodiment 2 of the present disclosure functions as a prediction device that predicts information related to a patient based on medical data of the patient. The prediction device adds a common layer and combines the common layer on a plurality of machine learning models based on a similarity between diseases for a pair of a prediction target and each disease. The common layer is trained by using training data sets related to a plurality of diseases. Thereby, effective training is performed by using as many training data sets as possible in consideration of the similarity between diseases.

### Exemplary Embodiment 3

Next, a prediction server 300 according to an exemplary embodiment 3 of the present disclosure will be described.

### (Prediction Server 300)

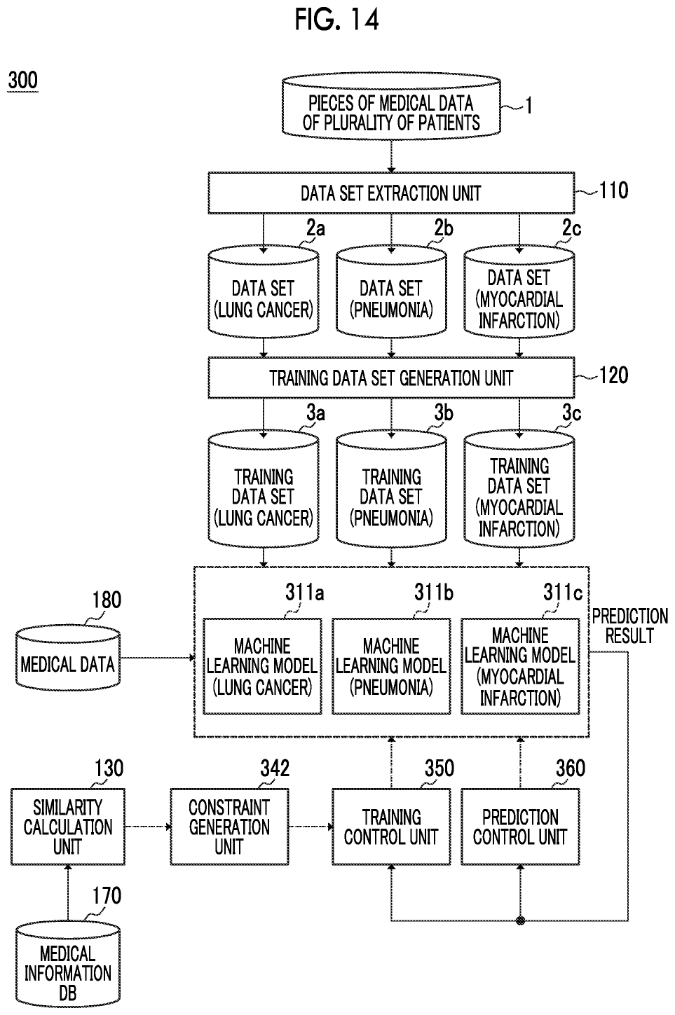

FIG. 14 is a diagram illustrating a configuration of a prediction server 300 according to an exemplary embodiment 3 of the present disclosure. The prediction server 300 includes a constraint generation unit 342 instead of the order determination unit 140 included in the prediction server 100 according to the exemplary embodiment 1. Further, in the prediction server 300, the training control unit 150 and the prediction control unit 160 included in the prediction server 100 according to the exemplary embodiment 1 are respectively replaced with a training control unit 350 and a prediction control unit 360.

In addition, the prediction server 300 includes machine learning models 311a to 311c specialized for each disease. Specifically, the machine learning model 311a is a model specialized in predicting a hospitalization period of a lung cancer patient. The machine learning model 311b is a model specialized in predicting a hospitalization period of a pneumonia patient. The machine learning model 311c is a model specialized in predicting a hospitalization period of a myocardial infarction patient.

### (Constraint Generation Unit 342)

The constraint generation unit 342 generates a constraint that is commonly applied in a case of training each machine learning model for each pair of the machine learning models 311a to 311c. The constraint is defined by the following expression.

L₁₂ (similarity between “lung cancer” and “pneumonia”, similarity between configurations of the machine learning models 311a and 311b)

L₂₃ (similarity between “pneumonia” and “myocardial infarction”, similarity between configurations of the machine learning models 311b and 311c)

L₃₁ (similarity between “myocardial infarction” and “lung cancer”, similarity between configurations of the machine learning models 311c and 311a)

In the expressions described above, the constraint L₁₂ has a smaller value as a positive correlation between the similarity between “lung cancer” and “pneumonia” and the similarity between configurations of the machine learning models 311a and 311b is larger. The constraint L₂₃ has a smaller value as a positive correlation between the similarity between “pneumonia” and “myocardial infarction” and the similarity between configurations of the machine learning models 311b and 311c is larger. The constraint L₃₁ has a smaller value as a positive correlation between the similarity between “myocardial infarction” and “lung cancer” and the similarity between configurations of the machine learning models 311c and 311a is larger.

As a specific function form of the constraint L₁₂=L₂₃=L₃₁=L(S1, S2), for example, the following function form can be given.

L(S1, S2)=−M log(|S1−S2|)

Here, S1 is a similarity between diseases, and S2 is a similarity between configurations of the machine learning models. In addition, A is a parameter for scale adjustment, and satisfies 0<λ<1. In addition, the similarity between configurations of the machine learning models can be defined as, for example, a distance or a cosine similarity between vectors having weights and biases of all neurons included in the machine learning models as components.

### (Training Control Unit 350)

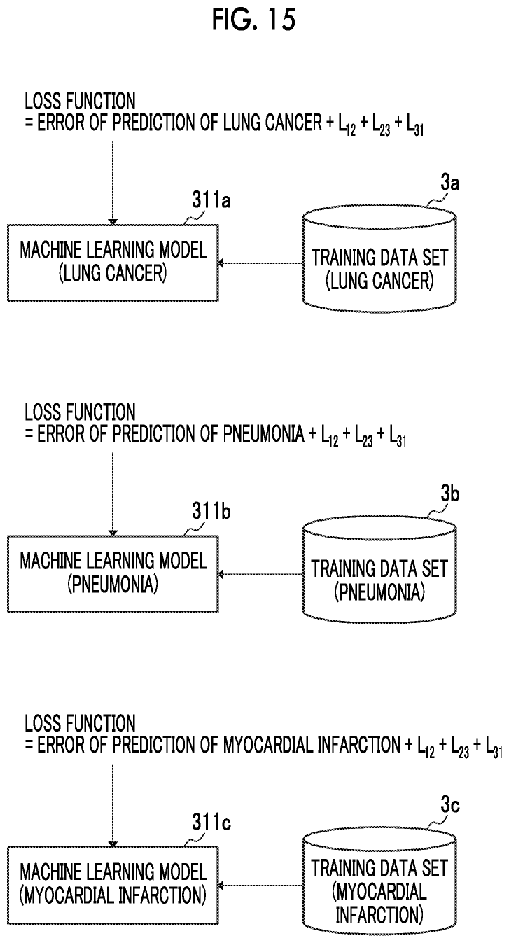

As illustrated in FIG. 15, the training control unit 350 trains the machine learning model 311a specialized for “lung cancer” by using the training data set 3a related to “lung cancer” according to an error backward propagation method. In this case, as a loss function, a function including the constraints L₁₂+L₂₃+L₃₁, in addition to an error between a prediction result and a correct answer label, is used. Thereby, training of the machine learning model 311a specialized for “lung cancer” is performed under a constraint that the similarity between a configuration of the machine learning model 311a and each of configurations of the other two machine learning models 311b and 311c and the similarity between “lung cancer” and each of “pneumonia” and “myocardial infarction” have a positive correlation.

Similarly, the training control unit 350 trains the machine learning model 311b specialized for “pneumonia” by using the training data set 3b related to “pneumonia” according to an error backward propagation method. In this case, as a loss function, a function including the constraints L₁₂+L₂₃+L₃₁, in addition to an error between a prediction result and a correct answer label, is used. Thereby, training of the machine learning model 311b specialized for “pneumonia” is performed under a constraint that the similarity between a configuration of the machine learning model 311b and each of configurations of the other two machine learning models 311c and 311a and the similarity between “pneumonia” and each of “myocardial infarction” and “lung cancer” have a positive correlation.

Similarly, the training control unit 350 trains the machine learning model 311c specialized for “myocardial infarction” by using the training data set 3c related to “myocardial infarction” according to an error backward propagation method. In this case, as a loss function, a function including the constraints L₁₂+L₂₃+L₃₁, in addition to an error between a prediction result and a correct answer label, is used. Thereby, training of the machine learning model 311c specialized for “myocardial infarction” is performed under a constraint that the similarity between a configuration of the machine learning model 311c and each of configurations of the other two machine learning models 311a and 311b and the similarity between “myocardial infarction” and each of “lung cancer” and “pneumonia” have a positive correlation.

As described above, since the loss function includes the constraints related to the correlation between the similarity between diseases and the similarity between the configurations of the models, training of each machine learning model indirectly depends on training of other machine learning models. This is based on an idea that, in a case where two diseases are similar, the configurations of two machine learning models specialized for the two diseases are also similar. Thereby, training is performed by indirectly using not only a training data set related to a specific disease but also a training data set related to another disease.

### (Prediction Control Unit 360)

In a case where it is desired to predict a hospitalization period of a lung cancer patient, the prediction control unit 360 inputs the medical data 180 of the lung cancer patient to the machine learning model 311a specialized for “lung cancer”.

In addition, in a case where it is desired to predict a hospitalization period of a pneumonia patient, the prediction control unit 360 inputs the medical data 180 of the pneumonia patient to the machine learning model 311b specialized for “pneumonia”.

In addition, in a case where it is desired to predict a hospitalization period of a patient with myocardial infarction, the prediction control unit 360 inputs the medical data 180 of the patient with myocardial infarction to the machine learning model 311c specialized for “myocardial infarction”.

As described above, the prediction server 300 according to the exemplary embodiment 3 of the present disclosure functions as a prediction device that predicts information related to a patient based on medical data of the patient. The prediction device generates, for each pair of the plurality of machine learning models, a constraint that a similarity between corresponding diseases and a similarity between configurations of the pair have a positive correlation. The training of each machine learning model is performed in consideration of the constraints. Thereby, effective training is performed by indirectly using not only a training data set related to a specific disease but also a training data set related to another disease.

Note that, in the above-described exemplary embodiments 1 to 3, an example in which the technical idea of the present disclosure is applied to a system that predicts a prognosis of an inpatient has been described. On the other hand, a scope to which the technical idea of the present disclosure can be applied is not limited thereto. For example, the technical idea of the present disclosure can be applied to a system that identifies a specific lesion in a medical image, a system that performs classification related to a specific disease, or the like.

Further, in the above-described exemplary embodiments 1 to 3, for example, as a hardware structure of a processing unit that executes various processing such as processing performed by the data set extraction unit, the training data set generation unit, the similarity calculation unit, the order determination unit, the common layer addition/combination unit, the constraint generation unit, the training control unit, and the prediction control unit, the following various processors may be used. Various processors include a programmable logic device (PLD) that is capable of changing a circuit configuration after manufacturing, such as a field-programmable gate array (FPGA), and a dedicated electric circuit that is a processor having a circuit configuration dedicatedly designed for executing specific processing, such as an application specific integrated circuit (ASIC), in addition to a CPU that is a general-purpose processor configured to execute software (program) to function as various processing units.

The various pieces of processing may be executed by one of the various processors or a combination of two or more processors of the same type or different types (for example, a plurality of FPGAs and a combination of CPU and FPGA). Further, the plurality of processing units may be configured by one processor. As an example where a plurality of processing units are configured with one processor, like system-on-chip (SOC), there is a form in which a processor that realizes all functions of a system including a plurality of processing units into one integrated circuit (IC) chip is used.

In this manner, the various processing units are configured by using one or more various processors as a hardware structure.

In addition, as the hardware structure of various processors, more specifically, an electric circuit (circuitry), in which circuit elements, such as semiconductor elements, are combined can be used.

Further, the technique of the present disclosure is applied to not only an operation program of a data merging rule generation device, an operation program of a learning device, and an operation program of an imaging device but also a non-transitory computer readable storage medium (a USB memory, a digital versatile disc (DVD)-read only memory (ROM), or the like) storing the operation program of the imaging device.

The entire disclosure of Japanese Patent Application No. 2021-137515 filed on Aug. 25, 2021 is incorporated into the present specification by reference.

All literatures, patent applications, and technical standards described in the present specification are incorporated in the present specification by reference to the same extent as in a case where the individual literatures, patent applications, and technical standards are specifically and individually stated to be incorporated by reference.

What is claimed is:

1. A prediction device that predicts information related to a patient based on medical data of the patient, the prediction device comprising:
a processor; and
a memory connected to or built in the processor, the processor being configured to execute:
training data set generation processing of generating M training data sets by classifying pieces of medical data of a plurality of patients into any one of M types of predetermined attributes;
similarity calculation processing of calculating, for each pair of the M types of attributes, a similarity between the attributes;
training processing of training one or a plurality of machine learning models based on the similarity between the attributes by using the M training data sets; and
prediction processing of causing the one or the plurality of machine learning models to predict the information related to the patient.

2. The prediction device according to claim 1, wherein:
the M types of attributes are M types of diseases or M types of medical departments, and
the similarity between the attributes is a similarity between the diseases or a similarity between the medical departments.

3. The prediction device according to claim 2, wherein the similarity between the attributes is calculated based on at least one of a distance between organs, a distance on a circulatory system, or a metastasis route of a cancer.

4. The prediction device according to claim 2, wherein:
the processor is configured to execute data set extraction processing of extracting M data sets by classifying pieces of medical data of a plurality of patients into any one of M types of predetermined attributes; and
the similarity between the attributes is calculated based on information included in the data set.

5. The prediction device according to claim 4, wherein the information included in the data set includes at least one of a symptom, an examination result, an examination image, an age of a patient, an attending physician, a medical department, a disease, a treatment, a medication, a candidate for differential diagnosis, or the number of co-occurrences.

6. The prediction device according to claim 1, wherein:
the one or the plurality of machine learning models are a single machine learning model,
the processor is further configured to execute order determination processing of determining an order of the M types of attributes based on a similarity between a prediction target and the attribute, and
the processor is configured to, in the training processing, train the single machine learning model by using the M training data sets related to the M types of attributes in order according to the order of the M types of attributes.

7. The prediction device according to claim 6, wherein the processor is configured to, in the order determination processing, set an attribute corresponding to the prediction target as an M-th attribute, and determine an order of the other M−1 attributes in descending order of a similarity between the M-th attribute and the attribute.

8. The prediction device according to claim 7, wherein, in the training processing, the processor is configured to:
set N to a natural number between 1 and M−1;
train the untrained single machine learning model by using the training data set related to an N-th attribute;
retrain the trained single machine learning model by using the training data set related to an (N+1)-th attribute; and
perform retraining in order to the M-th attribute.

9. The prediction device according to claim 1, wherein:
the one or the plurality of machine learning models include a plurality of machine learning models,
the processor is configured to further execute common layer addition/combination processing of adding a common layer and combining the common layer on the plurality of machine learning models based on a similarity between a prediction target and the attribute, and in the training processing, training of the common layer is performed by using training data sets related to a plurality of attributes.

10. The prediction device according to claim 1, wherein:
the one or the plurality of machine learning models include a first machine learning model related to a first attribute and a second machine learning model related to a second attribute,
the processor is configured to execute constraint generation processing of generating a constraint that a similarity between the attributes and a similarity between configurations of the first machine learning model and the second machine learning model have a positive correlation, and
in the training processing, training of the first machine learning model and the second machine learning model is performed in consideration of the constraint.

11. An operation method of a prediction device that predicts information related to a patient based on medical data of the patient, the method comprising:
a step of generating M training data sets by classifying pieces of medical data of a plurality of patients into any one of M types of predetermined attributes;
a step of calculating, for each pair of the M types of attributes, a similarity between the attributes;
a step of training one or a plurality of machine learning models based on the similarity between the attributes by using the M training data sets; and
a step of causing the one or the plurality of machine learning models to predict the information related to the patient.

12. A non-transitory computer readable medium storing a program for predicting information related to a patient based on medical data of the patient, the program causing a computer to execute a process comprising:
a step of generating M training data sets by classifying pieces of medical data of a plurality of patients into any one of M types of predetermined attributes;
a step of calculating, for each pair of the M types of attributes, a similarity between the attributes;
a step of training one or a plurality of machine learning models based on the similarity between the attributes by using the M training data sets; and
a step of causing the one or the plurality of machine learning models to predict the information related to the patient.
